(19) United States
(12) Patent Application Publication
(10) Pub. No.: US 2010/0166796 A1
(43) Pub. Date: Jul. 1, 2010

(54) METHOD OF INCREASING CELLULAR FUNCTION AND HEALTH OF GLUTATHIONE DEFICIENT ANIMALS

(76) Inventors: Robert Keller, Plantation, FL (US); Stacy L. O'Connor, Plantation, FL (US)

Correspondence Address: FROMMER LAWRENCE & HAUG, 745 FIFTH AVENUE- 10TH FL., NEW YORK, NY, 10151, US

(21) Appl. No.: 12/525,422

(22) PCT Filed: Jan. 31, 2008
(86) PCT No.: PCT/US08/52649

§ 371 Date: Jan. 25, 2010

Related U.S. Application Data
(60) Provisional application No. 60/887,382, filed on Jan. 31, 2007.

(51) Int. Cl. A61K 36/06; A61P 31/18

(52) U.S. Cl. 424/195.15

(57) ABSTRACT

This invention provides a method of improving glutathione (GSH) concentrations, both intra and extra-cellularly, in animals, thereby improving the cellular function of the immune and other bodily organ functions. The invention is directed toward a composition treating glutathione deficient animals which comprises N-acetylcysteine; vitamin C; L-glutamine; Silymarin; Cordyceps sp.; alpha-lipoic acid; and a pharmaceutically acceptable systemic carrier. The composition of the invention optionally comprises one or more of the following quercitin; N-acetyl-D-glucosamine; and dietary protein.

### INCORPORATION BY REFERENCE

Any foregoing applications and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### BACKGROUND OF THE INVENTION

1. Field of the Invention

This invention provides a method of improving glutathione (GSH) concentrations, both intra and extra-cellularly, in animals, thereby increasing the cellular function of the immune and other bodily organ functions. It comprises administration of a therapeutically effective amount of nutritional supplement which is composed of synergistic quantities of amino acids, peptides, bioflavanoids to promote the intracellular synthesis of glutathione by providing components for synthesis in an appropriate ratio; by increasing the function of enzymes involved in glutathione synthesis and recycling and by including a component to decrease inflammation by decreasing inflammatory cytokines (e.g. IL₁, IL₆) thereby decreasing free radical production and preserving the intra and extracellular stores of Glutathione. The compositions of the invention also serve to increase glutathione synthesis.

2. Brief Description of Related Art

Glutathione is a well-known tripeptide, which exists in two basic forms. The antioxidant form or “reduced glutathione” tripeptide is conventionally called “glutathione” and abbreviated as “GSH”. The oxidized form is a sulfur-sulfur linked compound known as glutathione disulfide (GSSG).

Glutathione in its biologically active, reduced form (GSH) has the formula:

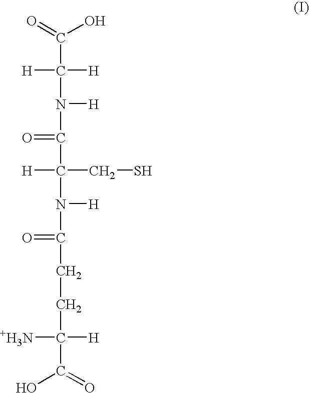

and is appropriately named γ-L-Glutamyl-L-cysteinyl glycine. It is ubiquitous in animals, plants, and microorganisms and being water soluble is found mainly in the cell cytosol and other aqueous phases of the living system. Glutathione often attains millimolar levels inside living cells, which makes it one of the most highly concentrated intracellular antioxidants.

Glutathione is homeostatically controlled, both inside the animal cell and outside. Enzyme systems synthesize it, utilize it, and regenerate it per the gamma-glutamyl cycle. (Meister A. Glutathione, Ascorbate and Cellular Protection Cancer Res (Suppl) 1994 (Apr. 1); 54:1969 S-1975S).

Glutathione is most concentrated in the animal liver (10 mM), where the P450 Phase II enzymes require it to convert fat-soluble substances into water-soluble GSH conjugates in order to facilitate their excretion. While providing GSH for their specific needs, the liver parenchymal cells export GSH to the outside, where it serves as systemic source of-SH/reducing power.

Briefly, glutathione synthesis occurs within animal cells in two closely linked enzymatically controlled reactions that utilize Adenosine Triphosphate (ATP) and draw on nonessential amino acids as substrates. First, cysteine and glutamate are combined (by the enzyme gamma-glutamyl cysteinyl synthetase, with availability of cysteine usually being the rate-limiting factor. Cysteine is generated from the essential amino acid methionine, from the degradation of dietary protein, or from turnover of endogenous proteins. The buildup of GSH acts to feedback-inhibit this enzyme, thereby helping to ensure homeostatic control over GSH synthesis.

The second GSH synthesis reaction combines gamma-glutamyl cysteine with glycine to generate GSH (catalyzed by GSH synthetase).

With regard to the essentiality of GSH for the survival of the animal, substantial information is available from studies on hereditary GSH depletion in the human, and from experimental depletion and repletion of GSH in animal models and cell cultures, see for example: Meister A. Larsson A. Glutathione Synthetase Deficiency and Other Disorders of the Gamma-Glutamyl Cycle; Scriver C R. et al eds. The Metabolic and Molecular Bases of Inherited Disease (Volume I). New York: McGraw-Hill: 1995:1461-1495 (Chapter 43); and Beutler E. Nutritional and Metabolic Aspects of Glutathione, Annu Rev Nutr 1989; 9:287-302.

Reduced GSH levels in animal cells are associated with a wide variety of pathophysiologic states, including hepatic dysfunction, malignancies, HIV infection, pulmonary disease, Parkinson's disease, related immunologic illnesses and pathophysiological conditions including every reported disease of aging; see for example the descriptions in Kidd, Alternative Medicine Review, Vol. 2, No. 3, pages 156-176 (1997).

The consequences of sustained GSH depletion are fatal. As cellular GSH is depleted, first individual cells die in those areas most affected. Then zones of tissue damage begin to appear. Localized free-radical damage spreads across the tissue in an ever-widening, self-propagating wave.

Previous attempts have been made to address deficient glutathione levels, for example U.S. RE 39,705. However, significant amounts of vitamin C were used in these formulations and the formulations did not adequately protect the functionality of glutathione or provide a proficient means of reducing inflammation, a cause of glutathione deficiency.

Therefore, a need still exists in the art to provide for alternatives in addressing deficient glutathione levels.

An object of this invention is to promote gastrointestinal absorption and intracellular uptake of components which will maximize intracellular reduced glutathione production by an animal including a human and reduce macrophage induced inflammation thereby reducing inflammatory cytokines and the resultant free radicals and thereby preserving glutathione concentration for its primary role in increasing mitochondrial ATP production by functioning as a general antioxidant for the redox reactions that occurs in the respiratory chain reactions. This object of the invention was achieved by making and using the compositions of the invention.

### SUMMARY OF THE INVENTION

The invention is directed toward a composition for treating glutathione deficient animals which comprises:
N-acetylcysteine;
vitamin C;
L-glutamine;
Silymarin
Cordyceps sp.;
alpha-lipoic acid; and
a pharmaceutically acceptable systemic carrier.

The composition of the invention optionally comprises one or more of the following:
quercitin;
N-acetyl-D-glucosamine; and
dietary protein.

The invention also comprises systemic administration of the composition of the invention to an animal suffering from low glutathione levels, to stimulate the natural production and recycling of glutathione and by decreasing inflammation and thus free radical production preserve glutathione concentrations both intra and extracellularly.

The term “low glutathione levels” as used herein means a blood glutathione level below about 440 μg glutathione/10¹⁰ erythrocytes, determined by the colorimetric method of Beutler et al., Improved Method for the Determination of Blood Glutathione, J. Lab. Clin. Med., 61; 882-8 (1963). Normal levels in humans ranges from about 440-654 μg/10¹⁰ erythrocytes.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right and hereby disclose a disclaimer of any previously described product, method of making the product or process of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

FIG. 1 depicts the effects on glutathione levels of an embodiment of the invention (MaxGXL™)—glutathione levels are measured in units of ng/10¹⁰ lymphocytes.

These and other embodiments are disclosed or are apparent from and encompassed by, the following Detailed Description.

### DETAILED DESCRIPTION OF THE INVENTION

Recently, there have been many scientific papers published discussing the direct relationship between decreased glutathione levels and the progression of many chronic diseases. Glutathione functions as an antioxidant, antitoxin and protector of red blood cells, and is extremely important to the immune system. It neutralizes free radicals minimizing the damage they cause and is profoundly important for cellular homeostasis.

As with other cell types, the proliferation, growth, and differentiation of immune cells is dependent on GSH. Both the T and the B lymphocytes require adequate levels of intracellular GSH to differentiate, and healthy humans with relatively low lymphocyte GSH were found to have significantly lower CD4 counts; Kinscherf R. Fischbach T. Mihm S. et al. Effect of glutathione depletion and oral N-acetylcysteine treatment on CD4+ and CD8+ cells. FASEB J 1994; 8:448-451. Intracellular GSH is also required for the T-cell proliferative response to mitogenic stimulation, for the activation of cytotoxic T “killer” cells, and for many specific T-cell functions, including DNA synthesis for cell replication, as well as for the metabolism of interleukin-2 which is important for the mitogenic response; Wu D. Meydani S N, Sastre J. et al and for protection against FAS mediated apoptosis; Morito, N., Yoh, K., Itoh, K., Nrf2 regulates the sensitivity of death receptor signals by affecting intracellular glutathione levels. Oncogene 22:9275, 2003. In-vitro glutathione supplementation enhances interleukin-2 production and mitogenic response of peripheral blood mononuclear cells from young and old subjects; J Nutr 1994; 124:655-663.

In summary, it has been demonstrated that decreased levels of glutathione may be a result of various types of prolonged stress, increased free radical formation and hyperactivity of the immune system. These factors in turn compromise the health of animal cells. Despite the apparent importance of adequate glutathione levels, little emphasis has heretofore been placed on replacing depleted stores. Some glutathione comes from the diet but the majority is made in the liver.

Studies have demonstrated that oral glutathione supplementation is not well absorbed by many of the animal's cells and does not replenish losses inside cells where it is most needed; Witschi A. Reddy S. Stofer B. et al. The systemic availability of Oral Glutathione. Eur. J. Clin. Pharmacol. 1992; 43:667-669.

The sulfur-containing amino acid 1-cysteine is the precursor that most limits the cellular biosynthesis of GSH. When substituted into the diet in place of the total protein allowance it was effective in raising GSH levels (see Witschi et al., supra.)

Glutathione esters, synthetic compounds prepared by linking the glycol end of GSH into ester bonds, have been the subject of much research by Meister, Anderson, supra., as potential oral GSH delivery compounds (see also U.S. Pat. No. 4,784,685). These esters do appear to be effective GSH delivery vehicles, but have the disadvantage that they yield alcohols in vivo when their ester bonds are broken, and their safety over the long term has yet to be satisfactorily demonstrated.

We have discovered that to efficiently raise the level of glutathione intracellularly, it is necessary to employ several different mechanisms that work simultaneously. First, essential elements needed by the body for the manufacture of glutathione must be introduced. Second, gastro-intestinal health of the animal must be optimal to facilitate nutrient absorption. Third, the liver function must be supported and protected as the liver is the glutathione “manufacturing and storage house”. Fourthly, recycling existing glutathione and enhancing enzymatic reactions that promote glutathione synthesis are also important functions which are advantageous to support. Lastly, Glutathione functions as an antioxidant to quench the detrimental effects (e.g. cellular function) of free radicals, which are obligate by products of cellular function in animals (oxygen fueled organisms). By decreasing inflammation specifically but not exclusively at the level of the macrophage, we reduce inflammatory cytokine and free radical productions, thereby preserving Glutathione for its primary use in quenching the oxidation products of the mitochondrial respiratory chain which facilitates maximum intracellular ATP production and thereby cellular function in every organ system in the body.

The invention is directed toward a composition treating glutathione deficient animals which comprises N-acetylcysteine; vitamin C; L-glutamine; silymarin; Cordyceps sp.; alpha-lipoic acid; and a pharmaceutically acceptable systemic carrier. The composition of the invention optionally comprises one or more of the following silymarin; quercitin; N-acetyl-D-glucosamine; and

dietary protein.

In one embodiment of the invention, the composition with a weight ratio which comprises:

N-acetylcysteine (about 10 to about 50);
Vitamin C (about 10 to about 30);
L-glutamine (about 20 to about 100);
Silymarin (about 0.5 to about 4);
Quercetin (about 1 to about 3);
alpha-lipoic acid (about 3 to about 9);
Cordyceps sp. (about 12 to about 36);
N-acetyl-D-glucosamine (about 5 to about 15); and
optionally a dietary protein in a weight ratio of about 1 to about 30.

In another embodiment of the invention, the composition with a weight ratio which comprises:

N-acetylcysteine (about 20 to about 40);
Vitamin C (about 15 to about 25);
L-glutamine (about 50 to about 70);
Silymarin (about 1 to about 3);
Quercetin (about 2 to about 4);
alpha-lipoic acid (about 4 to about 8);
Cordyceps sp. (about 20 to about 28);
N-acetyl-D-glucosamine (about 8 to about 12); and
optionally a dietary protein in a weight ratio of about 1 to about 30.

In still another embodiment of the invention, the composition with a weight ratio which comprises:

N-acetylcysteine (about 30);
Vitamin C (about 20);
L-glutamine (about 60);
Silymarin (about 2);
Quercetin (about 3);
alpha-lipoic acid (about 6);
Cordyceps sp. (about 24);
N-acetyl-D-glucosamine (about 10); and
optionally a dietary protein in a weight ratio of about 1 to about 30.

In another embodiment of the composition of the invention, the composition is free of probiotics.

In another embodiment of the composition of the invention, the amount of Vitamin C in the composition of the invention is less than 1000 mg. In another embodiment of this aspect of the invention, the amount of Vitamin C in the composition of the invention is less than 500 mg. In each embodiment, the minimum amount of Vitamin C present is at least 10 mg unless otherwise indicated.

Although not intuitively obvious, another mechanism of improving Glutathione concentrations intra and extracellularly could be achieved by reducing its ancillary utilization as a free radical trap in other oxidation reduction (redox) reactions thereby preserving it for use as a reductant for the oxidation reactions which occur during the production of ATP by the mitochondria of every animal cell (respiratory chain reactions). A preferred embodiment of this activity is Cordyceps sp standardized to >3% Cordycepin and/or 6% Cordycepic acid.

One substance noted to achieve this goal is Cordycepin, the active ingredient in the fungus, cordyceps sinensis (and other SP) (Shen, K. H., Lim, S. S., Lee, S. H., et al. Antioxidant and Immunostimlating Activities of the Fruiting Bodies of Paecilomyces Japonica, a new type of Cordyceps, Ann N.Y. Acad. Sciences 2001:928:261-73 and NG, T. B., Wan, H. X., Pharmacologic Actions of Cordyceps, a prized folk medicine, Pharm Pharmacol 2005: 57:1509-19.) although cordyceps has many demonstrated functions including, but not limited to, anti tumor, neuroprotective, and hypoglycemic effects, its functions of import to the present composition are its anti-inflammatory, immunodulatory, antioxidant and Hypolipdemic effects. Specifically it limits macrophage activation by suppression of nuclear factor Kappa beta. This obligately reduced production of proinflammatory cytokines including but not limited to IL₁ and IL₆; thereby reducing production of intra and extracellular free radicals and reducing the requirement for antioxidants. As Glutathione is the most prevalent intracellular antioxidant in animals and is also present extracellularly, this of necessity would increase Glutathione concentrations (Kim, H. G., Shrestha, B., Lim, S. Y., et al., Cordycepin inhibits lipopolysaccharide-induced inflammation by the suppression of NF-kappaB through AKT and p38 inhibition in RAW 264.7 macrophage cells. Eur J Pharmacol, 2006; 548:192-99. and Down-regulation of apoptotic and inflammatory genes by Cordyceps sinensis extract in rat kidney following ischemia/reperfusion. Shahed, A. R., Kim, S. I., and Shoskes, D. A., Transplant Proc, 2001; 33:2986-2987.) In addition to reducing its ancillary use in inflammation cordyceps, similar to silymarin enhances hepatic metabolism and ATP production thereby enhancing hepatic Glutathione production. (Manabe, N., Sugimoto, M., Azuma, Y., et al, Effects of the mycelial extract of cultured Cordyceps sinensis on in vivo hepatic energy metabolism in the mouse. Japan J. Pharmacol, 1996; 70: 85-88.)

Despite being at least additive, it not synergistic, the mechanisms of action of Cordyceps differs from the composition in claim 1 as Glutathione supplementation alone protects T-cells from receptor and chemical apoptosis (programmed cell death) but Cordyceps without Glutathione supplementation does not protect T-cells from either assault. (Cordyceps sinensis extracts do not prevent Fas-receptor and hydrogen peroxide-induced T-cell apoptosis. Buenz, E. J., Weaver, J. G, Bauer, B. A., et al. Ethnopharmacol, 2004; 90:57-62.) The composition herein, thereby increases Glutathione concentrations by promoting gastrointestinal absorption of the precursors, facilitates intracellular transport of the requisite components, promotes intracellular Glutathione synthesis, recycles oxidized Glutathione and preserves (protects) intra and extracellular concentrations of Glutathione by enhancing hepatic metabolism and thus hepatic Glutathione production (silymarin Cordyceps) and reducing ancillary Glutathione utilization by reducing macrophage induced inflammation, the production of pro inflammatory cytokines, and ultimately reducing the free radical production thus preserving/protecting Glutathione for its primary role in facilitating increased cellular energy, by enhancing mitochondrial ATP production.

The essential element needed by the animal cell to manufacture glutathione (GSH) is N-acetylcysteine (NAC). It has proven to be the most efficient dietary source of glutathione precursor. It is a precursor and the main limiting factor necessary for the body to manufacture reduced glutathione. NAC is well absorbed by the intestine and readily converted by the animal cell (particularly in the liver) to glutathione.

The absorption of N-acetylcysteine (NAC) and transport across the cellular membrane is facilitated by the presence of ascorbic acid (vitamin C). Vitamin C maximizes NAC transport across biological cell membranes and helps to conserve existing glutathione stores within the cell cytosol. Of greater importance, however, is Vitamin C function in recycling reduced (functional) Glutathione through induction of Glutathione Reductase.

L-glutamine is an essential dietary component for the support of gastrointestinal growth and function and it is utilized as fuel in the small intestines. It is used by the intestinal tract in large amounts for energy during periods of physiological stress. It has been shown to preserve liver glutathione after lethal hepatic injury and nourish tissues in the GI tract, liver and immune system, see for example; Souba, W. W., et al. The Role of Glutamine in Maintaining a Healthy Gut and Supporting the Metabolic Response to Injury and Infection. J. Of Surgical Res., 990:48(4):83-91.

The compounds to decrease inflammation in experimental systems by decreasing inflammatory cytokines include but are not limited to natural substances such as cordecepin, Reservatrol, green tea extracts, omega 3 fatty acids, beta glucans and any other natural substance which inhibit inflammation by blocking NF-κB activity.

As mentioned above, support of liver function in the animal being treated for low glutathione levels is advantageous. For this purpose, there may be orally administered to the animal the following:

Silymarin serves to improve and restore liver function. It quenches free radicals, reduces potential toxicity, and stimulates protein synthesis necessary to create new liver cells. Also known as “silibin”, “silybin” or “silybinin”, Silymarin is a generic term for extract from the mature fruits of Silybum marianum (sometimes Carduus marianus), commonly known as milk thistle; see Madaus AG publication: Legalon. Koln, Germany, 1989 and Valenzuela A, et al. Silymarin Protection Against Hepatic Lipin Peroxidation Induced by Acute Ethanol Intoxication in Rats, Biochemical Pharmacology, 1985:34(12):2209-2212. Silymarin is available under the trade name Legalon®., from Madaus AG, (Jarrow Formulas, Inc.; Madaus, 1989).

Quercetin [2-(3,4-Dihydroxyphenyl)-3,5,7-trihydroxy-4H-1-benzopyran-4-one] is used for its ability to eliminate toxic compounds found in the liver. It has anti-hepatotoxic, antiviral, anti-inflammatory and antibacterial properties. It may be synthesized by the method of Shakhova et al., Zh. Obsheh. Khim., 32, 390 (1962).

The utilization of N-acetylcysteine within the biological cell to synthesize glutathione is improved by the presence of alpha lipoic acid. Alpha lipoic acid increases the cell's ability to make glutathione. It enables the key enzyme required for glutathione synthesis to work under optimum conditions and induces a substantial increase in intracellular reduced glutathione; see Busse E. Zimmer G. Schopohl B, et al. Influence of alpha-lipoic acid on intracellular glutathione in vitro and in vivo; Arzneimittel-Forschung 1992; 42:829-831; and Han D. Handelman G. Marcocci, et al. Lipoic Acid Increases de novo Synthesis of Cellular Glutathione by Improving Cystine Utilization, Biofactors 1997; 6:321-338. 1995:29: 1263-73.

Advantageously, the following nutritionals are also employed in the method of the invention.

N-acetyl-D-glucosamine (NAG) is a key precursor in the biosynthesis of mucosal glycoproteins that form glycocalyx. The glycalyx is the most superficial, highly viscous layer of the gut mucosa that comes in contact with intestinal contents. The glycoprotein layer acts to protect the underlying tissues from exposure to enzymes, acid and bacterial assault while providing a selectively absorptive surface, Wilmore, D. W., et al, The gut: a Central Organ After Surgical Stress; Surgery 1988: 104, (5):917-23.

Furthermore, a source of dietary protein is preferred and advantageous to supplement the nutritional needs of the animal. We have found that the compositions of the invention and the method herein described are optimized by inclusion of a biologically active whey protein composition comprising an undenatured whey protein concentrate obtained from raw animal milk. This concentrate contains substantially all of the heat labile whey protein found in the raw milk. Representative of concentrate which are commercially available include Promod®., available from Ross Laboratories, Division of Abbott Laboratories, Chicago, Ill. Concentrates may also be prepared by the method described in U.S. Pat. No. 5,290,571, incorporated herein by reference thereto. The undenatured whey protein concentrates also contain a rich variety of immunoglobulins which boast the immunologic response of the animal treated with the concentrates; see for example U.S. Pat. No. 5,456,924 which is incorporated herein by reference thereto.

A high protein, low fat whey has immuno-supportive properties. It is rich in naturally active immunoglobulins, essential amino acids and other important nutrients critical for proper nutrient utilization within the gut.

We have discovered that the ingredients described above work synergistically to provide the necessary nutrients required for glutathione production while supporting the animal's ability to produce and preserve existing stores of GSH. The effect of the admixture of ingredients is far more significant than the individual ingredients alone.

This invention also relates also to pharmaceutical dosage unit forms for systemic administration (oral, topical administration) which are useful in treating animals, including humans. Formation of the dosage unit forms can be prepared using techniques known in the art such as those described in Remington—The Science and Practice of Pharmacy, 21^{st} Edition (2005), Goodman & Gilman's The Pharmacological Basis of Therapeutics, 11^{th} Edition (2005) and Ansel's Pharmaceutical Dosage Forms and Drug Delivery Systems (8^{th} Edition), edited by Allen et al., Lippincott Williams & Wilkins, (2005).

The term “dosage unit form” as used in this specification and in the claims refers to physically discrete units suitable as unitary dosage for animal subjects, each unit containing a predetermined quantity of the essential active ingredient, i.e. the composition of the invention; calculated to produce the desired effect in combination with the required pharmaceutical means which adapt said ingredient for systemic administration.

Examples of dosage unit forms in accordance with this invention are tablets, capsules, orally administered liquid preparations in liquid vehicles, suppositories, and dry preparations for the extemporaneous preparation of preparations in a liquid vehicle as well as liposomally encapsulated components as a vehicle for skin delivery.

Solid diluents or carriers for the solid oral pharmaceutical dosage unit forms are selected from the group consisting of lipids, carbohydrates, proteins and mineral solids, for example, starch, sucrose, kaolin, dicalcium phosphate, gelatin, acacia, corn syrup, corn starch, talc and the like.

Capsules, both hard and soft, are formulated with conventional diluents and excipients, for example, edible oils, talc, calcium carbonate, calcium stearate, magnesium stearate and the like. Liquid pharmaceutical preparations for oral administration may be prepared in water or aqueous solutions which advantageously contain suspending agents, such as for example, sodium carboxymethylcellulose, methylcellulose, acacia, polyvinyl pyrrolidone, polyvinyl alcohol and the like.

Skin delivery systems include compositions for topical delivery, percutaneous absorption or transdermal delivery. To enhance delivery of the compositions of the invention, the composition may optionally include a skin permeability enhancer compound or be delivered via iontophoresis or sonorphoresis.

Such preparations must be stable under the conditions of manufacture and storage, and ordinarily contain in addition to the basic solvent or suspending liquid, preservatives in the nature of bactericidal and fungicidal agents, for example, parabens, chlorobutanol, benzyl alcohol, phenol, thimerosal, and the like. In many cases it is preferable to include isotonic agents, for example, sugars or sodium chloride. Carriers and vehicles include vegetable oils, water, ethanol, and polyols, for example, glycerol, propylene glycol, liquid polyethylene glycol, and the like. Further improvement in Glutathione synthesis and function could be attained by adding Selenium, various B vitamins, Folic Acid and Vitamin D₃.

The pharmaceutical dosage unit forms are prepared in accordance with the preceding general description to provide an effective amount of the essential active ingredient per dosage unit form in admixture with the means for adaptation to systemic administration. In general, the unit dose form will contain 3 to 73 percent by weight of the essential active ingredient.

It will be appreciated that the exact dosage of the essential active ingredient constituting an effective amount for treatment of an animal according to the method of the invention will vary greatly depending on the specific nature of the clinical condition being treated, severity of the condition, species of animal, age, weight and condition of the animal, mode of administration of the dosage form and the specific formulation being administered. The exact dose required for a given situation may be determined by administration of a trial dose and observation of the clinical response. In general, an effective amount to be administered will be within a range of from about 0.1 mg. per kg. to about 50 mg. per kg. of body weight of the recipient, daily. Preferably 0.5 mg./kg. to about 25 mg./kg. daily is provided. In most instances, a single month of administration will effect a noticeable response and bring about the result desired. In cases such as the treatment of immunological conditions however, it may be desirable to repeat the administrations several times daily over longer periods of time.

The pharmaceutical compositions of the invention can be used to stimulate the natural production of glutathione in the biologically active cells of the animal and reduce symptoms of diseases caused by excess unneutralized free radicals. These diseases include but are not limited to the group consisting of pulmonary oxygen toxicity, adult respiratory distress syndrome, bronchopulmonary dysplasia, Chronic Obstructive Lung Disease, pulmonary fibrosis, sepsis syndrome, Parkinson's disease, Alzheimer's, autism and other neurodegenerative diseases including conditions of inflammatory dementia, encephalitis, endotoxemia, anoxia induced neuronal damage, ischemic reperfusion injury, inflammatory diseases, systemic lupus erythematosis, myocardial infarction, stroke, traumatic hemorrhage, spinal cord trauma, Crohn's disease, rheumatoid arthritis, diabetes, cataract formation, uvetis, macular degeneration, emphysema, gastric ulcers, oxygen toxicity, neoplasia, undesired cell apoptosis and radiation sickness.

The pharmaceutical compositions of the invention can be used to treat an animal suffering from one or more of the following illnesses from the group consisting of chronic viral infections: HIV, hepatitis C, chronic fatigue, immuno deficiency syndrome, immune deficiencies, cancer, B-cell malignancies, including lymphomas, chronic leukemia, myeloma Waldenstrom's and MGUS to improve immune defense productions and thereby mitigate the progression of the illnesses to thereby limit fatigue.

The pharmaceutical compositions of the invention can be used to promote the natural production of glutathione in the biologically active cells of the animal which accelerates the detoxification of ethanol and alleviates symptoms associated with excessive ethanol imbibations.

The pharmaceutical compositions of the invention can be used to promote the shift of the T-cell balance from TH2 to TH1 and decrease levels of IgE.

The pharmaceutical compositions of the invention can be used to decrease serum cholesterol and triglycerides.

The pharmaceutical compositions of the invention can be used to decrease fatigue, decrease the biologic effects of stress and/or increase energy and improve physical performance in an animal.

Another embodiment of the invention is directed to a method of improving function of the immune system in a patient in need thereof by increasing glutathione levels which comprises of administering a therapeutically effective amount of the composition of the invention.

Another embodiment of the method of improving function of the immune system is where the patient in need thereof is suffering from HIV, Hepatitis C, chronic fatigue syndrome or an acute viral infection.

In still another embodiment of the method of improving function of the immune system, glutathione levels are increased from about 100% to about 400% relative to glutathione levels prior to treatment with the composition of the invention. In one embodiment of the method of treatment, glutathione levels are increased from about 110% to about 320% relative to glutathione levels prior to treatment with the composition of the invention.

In yet another embodiment of the method of improving function of the immune system, glutathione levels remain increased at a level greater than 20%, after administration of the composition of the invention is terminated, relative to glutathione levels prior to treatment with the composition of the invention.

For the purpose of this invention, patients are inclusive of animals. Additionally, animals are intended to include but are not limited to mammals, birds and fishes. Mammals include but are not limited to humans, cats, dogs, cattle, chickens, cows, deer, goats, horses, llamas, pigs, sheep, yaks and zebras. Birds, include but are not limited to chickens, ostriches, quails and turkeys.

The following examples and preparations describe the manner and process of making and using the invention and set forth the best mode contemplated by the inventor of carrying out the invention but are not to be construed as limiting.

### Example 1

A mixture of the following ingredients is prepared by hand mixing:

| Ingredient | Weight Ratio |
|---|---|
| N-acetylcysteine | 30 |
| vitamin C | 20 |
| L-glutamine | 60 |
| silymarin | 2 |
| quercetin | 3 |
| alpha-lipoic acid | 6 |
| N-acetyl-D-glucosamine | 10 |
| Cordyceps sinesis | 24 |

The mixture constitutes the essential active ingredient of the invention, and may optionally be compounded together with a flavorant into wafers, tablets or capsules containing 750 to 14,000 mg of the essential active ingredient. In an uncompounded form, the powder dry mixture may be orally administered to a human (one teaspoonful, once or twice daily) as a dietary supplement or as recommended by a health care professional. Alternatively, the dry powder may be mixed with juice, water or food to facilitate administration. An embodiment of the mixture is known as MaxGXL™.

When administered to a human adult suffering from low levels of Glutathione (GSH) 1 to 6 dosage units daily, the level is adjusted upward to a normal range.

### Example 2

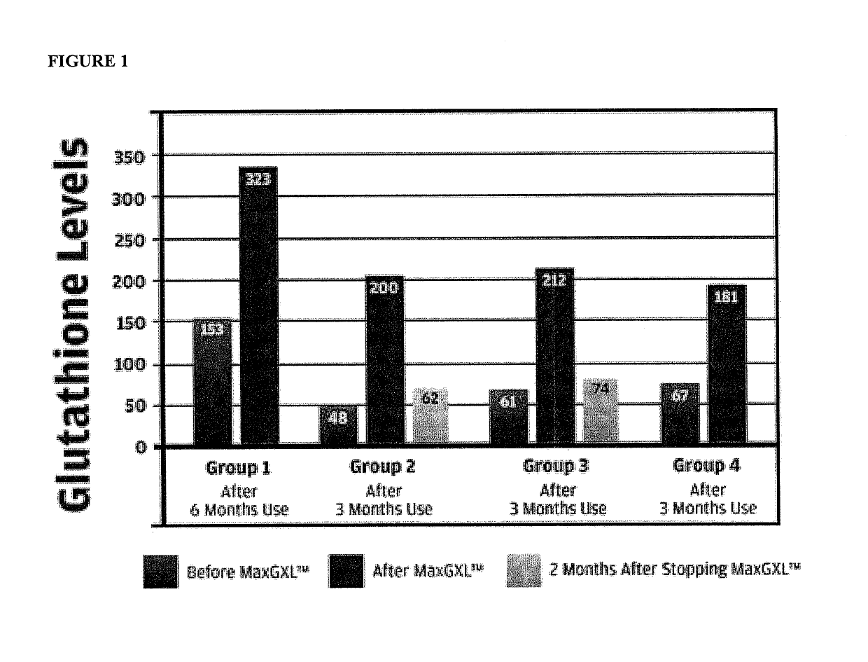

Our studies have shown that the administration of the a dosage unit (3 capsules) of the mixture of Example 1 from once to six times, preferably twice a day, is useful in the relief of immuno-deficiency in adult humans provoked by infective disease, or other etiological causes. For example, the composition of Example 1 can be used effectively to improve hepatic function e.g. decreased inflammation (ALT) in patients with chronic hepatitis C (see FIG. 1—Group 2 data) and patients who are receiving protease inhibitors as part of HAART therapy for HIV (see FIG. 1—Group 1). Both groups demonstrated an increase in intra lymphocyte GSH levels after the administration of the composition of Example 1.

The composition of Example 1 also displayed improved effects in patients with ME/CFS (chronic fatigue syndrome—FIG. 1—Group 3 data) and acute viral infection (FIG. 1—Group 4).

Glutathione measurement in lymphocytes is more physiologic than red blood cell measurements as lymphocyte levels correlate with functionality with 6-24 hours while red blood cell levels correlate with functionality between 90-120 days but would be considered to be predictive for addressing low glutathione levels in red blood cells.

The increases in glutathione levels was marked and represented an unexpected improvement in the art (Group 1 (107/153=111%); Group 2 (152-48=317%); Group 3 (151/61=248%); Group 4 (114/67=170%)).

In addition, the data from FIG. 1 shows that stoppage of treatment with the composition of Example 1 while resulting in significant decrease in glutathione levels (Group 2 (138/200=69%) and Group 3 (138/212=65%)), still resulted in an increase over pre-treatment levels (“residual increase”) after two months of treatment stoppage (Group 2 (14/48=29%) and Group 3 (13/61=21%)).

Other informal studies suggest that systemic administration of the composition results in an improvement in T lymphocyte function which correlates directly with an increased intra lymphocyte GSH. In addition, our data demonstrates that the inventive composition and method shifts the T-cell balance from TH2 (allergy producing) to TH1 (viral/tumor killing) and the increases intra lymphocyte GSH correlate directly with decreased levels of IgE the immunoglobulin associated with allergies.

Further informal studies suggest that:

Systemic administration of the composition increases natural killer cell function which is considered a primitive first line of cellular immune defense.

Systemic administration of the composition decreases serum cholesterol and triglycerides of between 10 and 20% in patients with a variety of hyperlipidemias and a decrease in myalgias associated with illness and exercise and improved muscle recovery after exercise.

Systemic administration of the composition decreases fatigue in patients suffering from a variety of illnesses including but not limited to chronic viral infections, HIV, hepatitis C, chronic fatigue, immunodeficiency syndrome, immune deficiencies, cancer, B-cell malignancies, including lymphomas, chronic leukemia, myeloma Waldenstrom's and MGUS. This makes the composition function as both a pharmaceutical and a therapeutic substance for patients suffering from the debilitating conditions.

As such, the combination formulated will improve hepatic function in conditions associated with chronic viral infections, as well as any condition associated with increased hepatic work and/or stress.

Furthermore, the composition as formulated, by increasing intracellular and extracellular Gluthatione levels should improve any clinical disease/condition associated with decreased Glutathione levels and/or inflammation

### Example 3

In a randomized placebo controlled double blind crossover test (a study type approved by the Institutional Review Board), the administration of a dosage unit (3 capsules) of the mixture of Example 1 which was administered to subject patients twice a day for two months. The patients were then subjected to a two-week washout period where no dosage was administered followed by a two-month period where a placebo was administered. Other subject patients were first administered the placebo then the mixture of Example 1 after the two-week washout period. The following observations were made:
1. The average increase in lymphocyte Glutathione levels while consuming the formulation encompassed by Example 1 was 250% (range 100%-400%) compared to their baseline and/or placebo values.

2. Indices of inflammation including Westegren Sedimentation Rate, C Reactive Protein, Cystatin (Kidney), TNF Alpha, and/or Adiponectin (liver) decreased 55% (range 20%-80%) while consuming the formulation encompassed by Example 1 compared to their baseline and/or placebo values.

3. In subjects with increased fasting Insulin (>10) and/or increased fasting Glucose, there was a significant decrease in both fasting Insulin and fasting Glucose while consuming the formulation encompassed by Example 1 compared to their baseline and/or placebo values.

4. There was a tendency toward decreased Cholesterol and LDL in subjects while consuming the preferred embodiment of the formulation compared to their baseline and/or placebo values.

5. In the self reported SF 32 Quality of Life Assessment Scale, the following trends were noted:
Improved concentration
Improved energy
Improved sleep
Decreased pain
Improved mood
Decreased irritability
in subjects while consuming the formulation encompassed by Example 1 compared to their baseline and/or placebo values.

Thus, by the present invention its advantages will be realized and although preferred embodiments have been disclosed and described in detail herein, its scope should not be limited thereby rather its scope should be determined by that of the appended claims.

What is claimed is:

1. A composition for treating glutathione deficient animals which comprises:
N-acetylcysteine;
vitamin C;
L-glutamine;
Silymarin;
Cordyceps sp.;
alpha-lipoic acid;
optionally further comprises one or more compounds selected from the group consisting of quercitin; N-acetyl-D-glucosamine; and dietary protein; and
a pharmaceutically acceptable systemic carrier.

2. The composition of claim 1, wherein the composition comprises:
N-acetylcysteine;
vitamin C;
L-glutamine;
Cordyceps sp.;
alpha-lipoic acid;
silymarin;
quercitin;
N-acetyl-D-glucosamine;
optionally a dietary protein; and
a pharmaceutically acceptable systemic carrier.

3. The composition of claim 2, wherein the composition has a weight ratio of:
N-acetylcysteine (about 10 to about 50);
Vitamin C (about 10 to about 30);
L-glutamine (about 20 to about 100);
Silymarin (about 0.5 to about 4);
Quercetin (about 1 to about 3);
alpha-lipoic acid (about 3 to about 9);
Cordyceps sp. (about 12 to about 36);
N-acetyl-D-glucosamine (about 5 to about 15); and
optionally a dietary protein in a weight ratio of about 1 to about 30.

4. The composition of claim 3, wherein the composition has a weight ratio of:
N-acetylcysteine (about 20 to about 40);
Vitamin C (about 15 to about 25);
L-glutamine (about 50 to about 70);
Silymarin (about 1 to about 3);
Quercetin (about 2 to about 4);
alpha-lipoic acid (about 4 to about 8);
Cordyceps sp. (about 20 to about 28);
N-acetyl-D-glucosamine (about 8 to about 12); and
optionally a dietary protein in a weight ratio of about 1 to about 30.

5. The composition of claim 4, wherein the composition has a weight ratio of:
N-acetylcysteine (about 30);
Vitamin C (about 20);
L-glutamine (about 60);
Silymarin (about 2);
Quercetin (about 3);
alpha-lipoic acid (about 6);
Cordyceps sp. (about 24);
N-acetyl-D-glucosamine (about 10); and
optionally a dietary protein in a weight ratio of about 1 to about 30.

6. The composition of claim 5, wherein the composition has no dietary protein.

7. The composition of claim 5, wherein the composition is probiotic free and contains less than 500 mg of Vitamin C.

8. A method of improving function of the immune system in a patient in need thereof by increasing glutathione levels which comprises of administering a therapeutically effective amount of the composition of claim 1.

9. The method of claim 8, wherein the patient in need thereof is suffering from HIV, Hepatitis C, chronic fatigue syndrome or an acute viral infection.

10. The method of claim 9, wherein the glutathione levels are increased from about 100% to about 400% relative to glutathione levels prior to administration of said composition.

11. The method of claim 9, wherein the composition has a weight ratio of:
N-acetylcysteine (about 20 to about 40);
Vitamin C (about 15 to about 25);
L-glutamine (about 50 to about 70);
Silymarin (about 1 to about 3);
Quercetin (about 2 to about 4);
alpha-lipoic acid (about 4 to about 8);
Cordyceps sp. (about 20 to about 28);
N-acetyl-D-glucosamine (about 8 to about 12); and
optionally a dietary protein in a weight ratio of about 1 to about 30.

12. The method of claim 11, wherein the composition has a weight ratio of:
N-acetylcysteine (about 30);
Vitamin C (about 20);
L-glutamine (about 60);
Silymarin (about 2);
Quercetin (about 3);
alpha-lipoic acid (about 6);
Cordyceps sp. (about 24);
N-acetyl-D-glucosamine (about 10); and
optionally a dietary protein in a weight ratio of about 1 to about 30.

13. The method of claim 12, wherein the composition has no dietary protein.

14. The composition of claim 12, wherein the composition is probiotic free and contains less than 500 mg of Vitamin C.
